(19) United States
(12) Patent Application Publication
(10) Pub. No.: US 2026/0131035 A1
(43) Pub. Date: May 14, 2026

(54) Mouse Anti-Human Monoclonal Antibody Against Glypican-3

(71) Applicants: University of Washington, Seattle, WA (US); Fred Hutchinson Cancer Center, North Seattle, WA (US)

(72) Inventors: James O. Park, Seattle, WA (US); Elizabeth Wayner, Seattle, WA (US)

(73) Assignees: University of Washington, Seattle, WA (US); Fred Hutchinson Cancer Center, North Seattle, WA (US)

(21) Appl. No.: 18/695,645

(22) PCT Filed: Sep. 30, 2022
(86) PCT No.: PCT/US2022/077438

§ 371 Date: Mar. 26, 2024

Related U.S. Application Data
(60) Provisional application No. 63/261,934, filed on Sep. 30, 2021.
(60) Provisional application No. 63/261,963, filed on Oct. 1, 2021.

(51) Int. Cl. A61K 51/10; A61P 35/00; C07K 16/30; G01N 33/57525; G01N 33/60

(52) U.S. Cl. CPC A61K 51/1093; A61K 51/1057; A61P 35/00; C07K 16/303; G01N 33/57525; G01N 33/60; A61K 2121/00; A61K 2123/00; C07K 2317/24

(57) ABSTRACT

Embodiments of the present disclosure provide an antibody comprising a light chain variable domain and a heavy chain variable domain. The light chain variable domain comprises a CDRL1 with an amino acid sequence as set forth in SEQ ID NO:8; a CDRL2 with an amino acid sequence as set forth in SEQ ID NO:10; and a CDRL3 with an amino acid sequence as set forth in SEQ ID NO:12. The heavy cham variable domain comprises a CDRH1 with an amino acid sequence as set forth in SEQ ID NO:4; a CDRH2 with an amino acid sequence as set forth in SEQ ID NO:16; and a CDRH3 with an amino acid sequence as set forth in SEQ ID NO:18; wherein the antibody specifically binds glypican-3 (GPC3).

### CROSS-REFERENCES TO RELATED APPLICATIONS

This application claims the benefit of U.S. Provisional Application No. 63/261,934, filed Sep. 30, 2021, and U.S. Provisional Application No. 63/261,963, filed Oct. 1, 2021, the contents of which are incorporated herein by reference in their entireties.

### STATEMENT REGARDING SEQUENCE LISTING

The Sequence Listing XML associated with this application is provided in XML format and is hereby incorporated by reference into the specification. The name of the XML file containing the sequence listing is 3915-P1218WOUW_Seq_List_20220929_ST26 The XML file is 16 KB; was created on Sep. 29, 2022; and is being submitted via Patent Center with the filing of the specification.

### BACKGROUND

Hepatocellular carcinoma (HCC) is a significant cause of morbidity and mortality worldwide, particularly in advanced stages. Although novel combination therapies are being developed (e.g., immune checkpoint and tyrosine kinase inhibitors), these regimens are limited by modest efficacy and significant adverse effects. New therapies for treatment of advanced HCC are needed.

Targeted alpha particle therapy (TAT) is a promising new class of cancer therapies which create double-stranded DNA breaks via a high linear energy transfer and induce cytotoxic T lymphocyte driven antitumor immune response. Thorium-227 (²²⁷Th, t_{1/2} 18.7 d) is an alpha emitting radionuclide that has been attached to monoclonal antibody conjugates using bifunctional octadentate ligands, such as isothiocyanato-benzyl-DOTA and 3,2-hydroxypyridinone (3,2-HOPO) derivatives. While alpha-emitters are generally appealing because of the higher radiation payload deposited over shorter distances (high LET), minimizing the risk of off-target toxicity, the half-life of ²²⁷Th makes it appealing for the treatment of solid tumors.

Thorium-227-labeled antibody conjugates are currently being evaluated in treatment of several malignancies including acute myeloid leukemia, multiple myeloma, renal cell carcinoma, non-Hodgkin's lymphoma, mesothelin-positive mesothelioma and ovarian cancer. Glypican-3 (GPC3) is a cell surface glycoprotein highly expressed in hepatocellular carcinomas (HCC) making it a promising target for novel therapeutic and diagnostic applications.

Accordingly, there remains a need to develop thorium-227-labeled antibody conjugates that target GPC3 for the treatment of HCC. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter

In accordance with the foregoing, in one aspect of the invention, the disclosure provides an antibody comprising a CDRL1 with an amino acid sequence as set forth in SEQ ID NO:8; a CDRL2 with an amino acid sequence as set forth in SEQ ID NO:10; and a CDRL3 with an amino acid sequence as set forth in SEQ ID NO:12, and a heavy chain variable domain comprising: a CDRH1 with an amino acid sequence as set forth in SEQ ID NO:4; a CDRH2 with an amino acid sequence as set forth in SEQ ID NO:16; and a CDRH3 with an amino acid sequence as set forth in SEQ ID NO:18; wherein the antibody specifically binds glypican-3 (GPC3). In some embodiments, the antibody is conjugated to a radioisotope. In some embodiments, the radioisotope is thorium-227. In other embodiments, the thorium-227 is conjugated to the antibody via p-SCN-BN-H4octapa-NCS. In still other embodiments, the antibody is conjugated to p-SCN-BN-H4octapa-NCS.

In another aspect of the invention, the disclosure provides for a pharmaceutical composition comprising the antibody described above.

In another aspect of the invention, the disclosure provides for a nucleic molecule comprising a nucleotide sequence encoding the amino acid sequence of the antibody described above.

In another aspect of the invention, the disclosure provides for a method of detecting the presence of glypican-3 (GPC3) on a cell or tissue, the method can comprise contacting the cell or tissue with the antibody described above and detecting the binding of the antibody to the cell or tissue.

In another aspect of the invention, the disclosure provides for a method of imaging the presence or distribution of glypican-3 (GPC3) in a subject, the method can comprise administering the pharmaceutical composition described above to the subject.

In another aspect of the invention, the disclosure provides a method of treating hepatocellular carcinoma (HCC) in a subject in need thereof, the method comprising administering an effective amount of the pharmaceutical composition described above to the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1: Illustration of the decay scheme of ²²⁷Th.

FIG. 2: Conjugation with octapa to αGPC3. Stained isoelectric focusing gel showing the pI changes in the unconjugated αGPC3 mAb (right lane), when 5, 10 and 15 Eq of the octapa-NCS reagent are reacted with αGPC3 mAb.

FIG. 3: ²²⁷Th-labeled mAbs are stable in vitro. Shown is the percentage bound thorium of αBHV1-octapa and αGPC3-octapa incubated in PBS over 14 d with and without ascorbic acid. *AA is ascorbic acid

FIG. 4: αGPC3-octapa maintains binding affinity for GPC3 in vitro. In vitro GPC3 binding assessed by flow cytometry on human HepG2 cells with unconjugated, and αGPC3-octapa compared to unstained control (black). Three biologic replicates samples shown.

FIGS. 5A through 5C: Comparative biodistribution of ²²⁷Th-octapa-αGPC3 and ²²⁷Th-octapa-αBHV1. 5A) Tissue biodistribution of ²²⁷Th-octapa-αGPC3 in tumor bearing mice 1 d (n=7), 7 d (n=7) and 23 d (n=6) after injection. 5B) ²²⁷Th accumulation in tumor tissue and 5C) the tumor to liver ratio 1 d (n=7), 7 d (n=5 BHV1, n=3 GPC3) and 23 d (n=4) after injection with ²²⁷Th-octapa-αBHV1 or ²²⁷Th-octapa-αGPC3. Bar denotes mean, error bar denotes standard deviation.

FIGS. 6A and 6B: Comparative blood clearance of ²²⁷Th radiolabeled αGPC3-octapa and of αBHV1-octapa. Comparative blood clearance profiles in tumor bearing mice at 5, 15, 30, 60, 120, 240 min, 24 h, 7 d and 23 d after injection with ²²⁷Th radiolabeled αGPC3-octapa and αBHV1-octapa (n=4/time point). Symbol denotes mean, error bar denotes standard deviation.

FIG. 7: ²²⁷Th-octapa-GPC3 reduces tumor burden in murine model. Serum AFP before (day −5) and 23 days after receiving no treatment (n=9), or tail vein injection of 500 kBq/kg ²²⁷Th-octapa-αBHV1 (n=9), 250 kBq/kg (n=10) or 500 kBq/kg ²²⁷Th-octapa-αGPC3 (n=12). Bar represents mean. Symbols denote individual mice. *denotes p<0.05 after unpair two-way ANOVA with Sidak multiple comparison test.

FIG. 8: Schematic representation of an embodiment of the method.

FIG. 9A through 9D: Sequence alignments for αGPC3 monoclonal antibody variable regions according to certain embodiments of the disclosure. FIG. 9A. Sequence alignment of nucleic acids encoding αGPC3 monoclonal antibody light chain variable regions for two isolates. FIG. 9B. Sequence alignment of amino acids of two αGPC3 monoclonal antibody light chain variable regions for the same isolates described in FIG. 9A. FIG. 9C. Sequence alignment of nucleic acids encoding αGPC3 monoclonal antibody heavy chain variable regions for two isolates. FIG. 9D. Sequence alignment of amino acids of two αGPC3 monoclonal antibody heavy chain variable regions for the same isolates described in FIG. 9C. In each alignment, the top line is the CB isolate and the bottom line is the UW isolate. Discrepancies are shaded. Complementarity determining regions (CDRs) are indicated in underlined.

FIG. 10: chemical structure of octapa.

FIG. 11: In vitro GPC3 binding assessed by flow cytometry on human HepG2 cells with unconjugated and αGPC3-octapa 5 eq, αGPC3-octapa 10 eq, αGPC3-octapa 15 eq.

FIG. 12: Tissue biodistribution of ²²⁷Th-octapa-αBHV1 in tumor bearing mice 1 d (n=7), 7 d (n=7) and 23 d (n=6) after injection.

FIGS. 13A through 13I: Serum chemistry profiles of animals 23 d after receiving no treatment, 500 kBq/kg ²²⁷Th-αBHV1, 250 kBq/kg ²²⁷Th-αGPC3 or 500 kBq/kg ²²⁷Th-αGPC3. Line represents mean. Symbols denote individual mice.

### DETAILED DESCRIPTION

Hepatocellular carcinoma (HCC) is a significant cause of morbidity and mortality worldwide with limited therapeutic options for advanced disease. Targeted alpha therapy (TAT) is an emerging class of targeted cancer therapy in which alpha-particle-emitting radionuclides, such as thorium-227, are specifically delivered to cancer tissue. Glypican-3 (GPC3) is a cell surface glycoprotein highly expressed on HCC. This disclosure describes the development and in vivo efficacy of a ²²⁷Th-labeled GPC3 targeting antibody conjugate (²²⁷Th-octapa-αGPC3) for treatment of HCC in an orthotopic murine model. Briefly, as described in more detail below, the chelator p-SCN-Bn-H₄octapa-NCS (octapa) was conjugated to a GPC3 targeting antibody (αGPC3) for subsequent ²²⁷Th radiolabeling (octapa-αGPC3). Conditions were varied to optimize radiolabeling of ²²⁷Th. In vitro stability was evaluated by measuring percentage of protein-bound ²²⁷Th by gamma-ray spectroscopy. An orthotopic athymic Nu/J murine model using HepG2 cells was developed. Biodistribution and blood clearance of ²²⁷Th-octapa-αGPC3 was evaluated in tumor bearing mice. Efficacy of ²²⁷Th-octapa-αGPC3 was assessed in tumor bearing animals with serial measurement of serum alpha-fetoprotein at 23 days after radionuclide injection.

Octapa-conjugated αGPC3 provided up to 70% ²²⁷Th labeling yield in 2 h at room temperature. In the presence of ascorbate, ≥97.8% of ²²⁷Th was bound to GPC3-octapa after 14 d in phosphate buffered saline. In HepG2 tumor-bearing mice, highly specific GPC3 targeting was observed, with significant ²²⁷Th-octapa-αGPC3 accumulation in the tumor over time and minimal accumulation in normal tissue. 23 days after treatment, significant reduction in tumor burden was observed in mice receiving 500 kBq/kg ²²⁷Th-octapa-αGPC3 by tail vein injection. No acute off-target toxicity was observed and no animals died prior to termination of the study. ²²⁷Th-octapa-αGPC3 was observed to be stable in vitro, maintain high specificity for GPC3 with favorable biodistribution in vivo, and result in significant antitumor activity without significant acute off-target toxicity in an orthotopic murine model of HCC.

In accordance with the foregoing, in one aspect the disclosure provides an antibody that specifically binds glypican-3 (GPC3).

As used herein, the term “antibody” is used herein in the broadest sense and encompasses various immunoglobulin-based structures derived from any antibody-producing mammal (e.g., mouse, rat, rabbit, and primate, including human), and which specifically bind to an antigen of interest (e.g., GPC3). The term refers to a molecule that incorporates one or more antibodies or antigen-binding fragments thereof and may optionally incorporate alterations and/or additional elements. An antibody fragment specifically refers to an intact portion or subdomain of a source antibody that still retains antigen-binding capability. Often, antibody derivatives incorporate at least some additional modification in the structure of the antibody or fragment thereof, or in the presentation or configuration of the antibody or fragment thereof. Exemplary antibodies of the disclosure include polyclonal, monoclonal and recombinant antibodies. Exemplary antibodies or antibody derivatives of the disclosure also include multi-specific antibodies (e.g., bispecific antibodies), humanized antibodies, murine antibodies, chimeric, mouse-human, mouse-primate, primate-human, etc.

In some embodiments, the disclosed antibodies are monoclonal antibodies. Monoclonal antibodies can be produced using hybridoma methods (see. e.g., Kohler, B. and Milstein, C. (1975) Nature 256:495-497 or as modified by Buck, D. W., et al., In Vitro, 18:377-381(1982). In some embodiments, the antibody of interest can be sequenced, and the polynucleotide sequence may then be cloned into a vector for expression or propagation. The sequence encoding the antibody of interest can be maintained in the vector in a host cell, and the host cell can then be expanded and frozen for future use.

As indicated, antibodies can be further modified to created derivatives that suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin. Any of these antibodies, including fragments, thereof, are encompassed by the disclosure. Antibody fragments and derivatives that recognize specific epitopes can be generated by any technique known to those of skill in the art, including proteolytic cleavage of immunoglobulin molecules using enzymes such as papain or pepsin. Further, the antibodies or fragments thereof of the present disclosure can also be generated using various phage display methods known in the art. Finally, the antibodies or fragments thereof can be produced recombinantly according to known techniques.

The disclosed antibody comprises a light chain variable domain and a heavy chain variable domain. The light chain variable domain comprises: a CDRL1 with an amino acid sequence as set forth in SEQ ID NO:8, a CDRL2 with an amino acid sequence as set forth in SEQ ID NO:10, and a CDRL3 with an amino acid sequence as set forth in SEQ ID NO-12. The heavy chain variable domain comprises: a CDRH1 with an amino acid sequence as set forth in SEQ ID NO:4, a CDRH2 with an amino acid sequence as set forth in SEQ ID NO:16, and a CDRH3 with an amino acid sequence as set forth in SEQ ID NO:18.

In some embodiments, the antibody comprises a variable light chain region comprising an amino acid sequence with at least about 90% identity, e.g., about 90%, 91%, 92%, 93%, 94%, 95%, 96%, 97%, 98%, 99%, or 100%, to the sequence set forth in SEQ ID NO:2 or SEQ ID NO:4. In some embodiments, the antibody comprises a variable heavy chain region comprising an amino acid sequence with at least about 90% identity. e.g., about 90%, 91%, 92%, 93%, 94%, 95%, 96%, 97%, 98%, 99%, or 100%, to the sequence set forth in SEQ ID NO:6.

In some embodiments, the antibody is a monoclonal antibody. In some embodiments, the antibody is a humanized antibody. In some embodiments, the antibody is conjugated to a radioisotope. e.g., thorium-227. In some embodiments, the thorium-227 is conjugated to the monoclonal antibody via p-SCN-BN-H4octapa-NCS.

In some embodiments, the antibody is conjugated to p-SCN-BN-H4octapa-NCS, thus providing a capacity to further conjugate a radioisotope, e.g., thorium-227.

In another aspect, the disclosure provides a pharmaceutical composition comprising the antibody. The composition can further comprise a pharmaceutically acceptable carrier, which can be readily designated by persons skilled in the art for the intended pharmaceutical purpose.

In another aspect, the disclosure provides a method of treating a hepatocellular carcinoma (HCC) in a subject in need thereof. The method comprises administering an effective amount of the pharmaceutical composition described herein to the subject. In some embodiments, the subject is human. The term “treating” and grammatical variants thereof may refer to any indicia of success in the treatment or amelioration or prevention of a disease or condition (e.g., HCC), including any objective or subjective parameter such as reduction in GPC3+ cells, slowing of growth of GPC3+ cells, abatement, remission, diminishing of symptoms or making the disease condition more tolerable to the patient, slowing in the rate of degeneration or decline, or making the final point of degeneration less debilitating. The treatment or amelioration of symptoms can be based on objective or subjective parameters, including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., HCC), or any combination thereof. The term “therapeutic effect” refers to the reduction, elimination, or prevention of the disease or condition, symptoms of the disease or condition, or side effects of the disease or condition in the subject.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used here, the term “pharmaceutically acceptable carrier” means a pharmaceutically-acceptable material, composition or vehicle, such as a liquid or solid filler, diluent, excipient, manufacturing aid or solvent encapsulating material necessary or used in formulating an active ingredient or agent for delivery to a subject. Each carrier must be “acceptable” in the sense of being compatible with the other ingredients of the formulation and not injurious to the patient. Embodiments of pharmaceutically acceptable carriers can be any carrier well-known in the art. One of ordinary skill in the art will be familiar with pharmaceutically acceptable carriers and can select the type of carrier and the amount of carrier appropriate for method.

In some embodiments, the pharmaceutical composition can comprise the antibody described herein combined with a pharmaceutically acceptable carrier, wherein the antibody and the carrier are combined in a container. In other embodiments, the pharmaceutical composition can comprise the antibody described herein in a first container and the pharmaceutically acceptable carrier in a second container, wherein the first and second container are administered for a therapeutic purpose. In some embodiments, the first container is administered before the second container. In other embodiments, the first container is administered after the second container. In still other embodiments, the first container is administered concurrently with the second container. In still other embodiments, the antibody as described herein can be co-administered with a second therapeutic for the treatment of hepatocellular carcinoma (HCC). The second therapeutic for the treatment of HCC can be any therapeutic agent well-known and commonly used for the treatment of HCC. By “co-administer” it is meant that the antibody of the present invention is administered at the same time, just prior to, or just after the administration of the second therapeutic.

As used the term “administering” includes oral administration, intravenous, intraperitoneal, intramuscular, intralesional, or subcutaneous administration to a subject. Administration is by any route, including parenteral and transmucosal (e.g., buccal, sublingual, palatal, gingival, nasal, vaginal, rectal, or transdermal). Parenteral administration includes. e.g., intravenous, intramuscular, intra-arteriole, intradermal, subcutaneous, intraperitoneal, intraventricular, and intracranial.

In another aspect, the disclosure provides a nucleic acid molecule comprising a nucleotide sequence encoding the amino acid sequence of the monoclonal antibody described above. In some embodiments the nucleic acid is characterized by one or more (e.g., 2, 3, 4, 5, or all) of the following: wherein the CDRL1 is encoded by a nucleic acid sequence that encodes the amino acid sequence as set forth in SEQ ID NO:8, wherein the CDRL2 is encoded by a nucleic acid sequence that encodes the amino acid sequence as set forth in SEQ ID NO:10, wherein the CDRL3 is encoded by a nucleic acid sequence that encodes the amino acid sequence as set forth in SEQ ID NO:12, wherein the CDRH1 is encoded by a nucleic acid sequence that encodes the amino acid sequence as set forth in SEQ ID NO:14, wherein the CDRH2 is encoded by a nucleic acid sequence that encodes the amino acid sequence as set forth in SEQ ID NO:16, and wherein the CDRH3 is encoded by a nucleic acid sequence that encodes the amino acid sequence as set forth in SEQ ID NO:18. In some embodiments, the nucleic acid is characterized by one or more (e.g., 2, 3, 4, 5, or all) of the following: wherein the CDRL1 is encoded by a nucleic acid sequence as set forth in SEQ ID NO:7, wherein the CDRL2 is encoded by a nucleic acid sequence as set forth in SEQ ID NO:9, wherein the CDRL3 is encoded by a nucleic acid sequence as set forth in SEQ ID NO:11, wherein the CDRH1 is encoded by a nucleic acid sequence as set forth in SEQ ID NO:13, wherein the CDRH2 is encoded by a nucleic acid sequence as set forth in SEQ ID NO:15, and wherein the CDRH3 is encoded by a nucleic acid sequence as set forth in SEQ ID NO:17.

In another aspect, the disclosure also provides a vector comprising the nucleic acid described above and a promoter operatively linked to the nucleic acid.

In another aspect, the disclosure also provides a method of detecting the presence of glypican-3 (GPC3) on a cell or tissue, comprising contacting the cell or tissue with the antibody as described herein and detecting binding of the antibody to the cell or tissue. In some embodiments, the cell is a liver cell or the tissue is a liver tissue. In some embodiments, the cell or tissue is in vitro in a biological sample obtained from a subject. In some embodiments, the cell or tissue is in vivo in a subject. In some embodiments, the detection of binding of the antibody to the cell or tissue indicates that the subject has hepatocellular carcinoma (HCC).

In another aspect, the disclosure provides a method of imaging the presence or distribution of glypican-3 (GPC3) in a subject. The method comprises administering the pharmaceutical composition described above to the subject and permitting the antibody to bind to GPC3 present in the cell.

In some embodiments, the antibody as described herein is conjugated to an imaging agent to enable detection of antibody binding to the cell or tissue of interest (e.g., GPC3 cell). In some embodiments, the imaging agent can include but is not limited to radionuclides, detectable tags, fluorophores, fluorescent proteins, enzymatic proteins, and the like. One of skill in the art will be familiar with imaging agents and can select a well-known imaging agent according to the type and location of the cell to be imaged. Additionally, one skilled in the art can use any well-known imaging techniques to detect binding of the antibody to the cell and/or tissue of interest.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press. Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons. New York (2010) Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed in this description and/or the claims are provided here. The definitions are provided to aid in describing particular embodiments and are not intended to limit the claimed subject matter, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising.” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

Reference to sequence identity addresses the degree of similarity of two polymeric sequences, such as protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (e.g., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

This Example discloses the development of ²²⁷Th-labeled GPC3 antibody conjugate (²²⁷Th-octapa-αGPC3) and evaluation of the radiolabeling properties, in vivo biodistribution and efficacy in a GPC3-positive hepatic orthotopic xenograft murine model of HCC.

Conjugation of αGPC3 and αBHV1 with Octapa

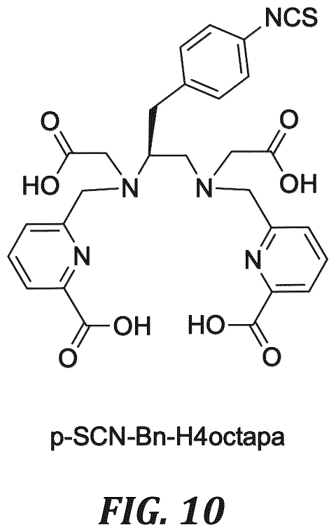

Reagents and solvents purchased from commercial sources were analytical grade or higher and were used without further purification unless noted. High purity nitric acid was obtained from Honeywell Fluka as TraceSELECT™ Ultra grade. Antibodies were demetallated prior to and after octapa-NCS conjugation as previously described (Ludwig A D, Labadie K P, Seo Y D, et al. Yttrium-90-Labeled Anti-Glypican 3 Radioimmunotherapy Halts Tumor Growth in an Orthotopic Xenograft Model of Hepatocellular Carcinoma. J Oncol. 2019; 2019:4564707). Pipette tips, syringes and plastic vials used for monoclonal antibody (mAb) conjugation and ²²⁷Th labeling were acid-washed to rid the surface of trace metals. [²²⁷Th]Th(NO₃)₄ was purchased from Oak Ridge National Laboratory (ORNL) and was purified from its decay daughters prior to use (Ferrier M G, et al. Thorium chelators for targeted alpha therapy: Rapid chelation of thorium-226. J Labelled Comp Radiopharm. 2020). The bifunctional chelator p-SCN-Bn-H₄octapa (octapa) was synthesized at the University of British Columbia (chemical structure shown in FIG. 10) (Price E W, et al. H4octapa: an acyclic chelator for 111In radiopharmaceuticals. J Am Chem Soc 2012, 134.8670-8683; Price E W, et al. H(4)octapa-trastuzumab: versatile acyclic chelate system for 11 In and 177Lu imaging and therapy. J Am Chem Soc. 2013; 135:12707-12721). The anti-GPC3 antibody (αGPC3) was generated and produced at Fred Hutchinson Research Center antibody core facility as previously described (Sham J G, et al. Glypican-3-targeted 89Zr PET imaging of hepatocellular carcinoma. J Nucl Med. 2014; 55:799-80; Wayner E A. Hoffstrom B G. Development of monoclonal antibodies to integrin receptors. Methods Enzymol. 2007; 426:117-153). The isotype control (IgG₁) antibody against bovine herpes virus, αBHV1 was a generous gift from the Orozco laboratory (Pagel J M, et al. Pretargeted radioimmunotherapy using anti-CD45 monoclonal antibodies to deliver radiation to murine hematolymphoid tissues and human myeloid leukemia. Cancer Res. 2009; 69:185-192). The conjugation reaction was previously described (Price E W. et al. H4octapa: an acyclic chelator for 111In radiopharmaceuticals. J Am Chem Soc. 2012; 134:8670-8683; Price E W, et al. H(4)octapa-trastuzumab: versatile acyclic chelate system for 111In and 177Lu imaging and therapy. J Am Chem Soc. 2013; 135:12707-12721).

Size-exclusion high-performance liquid chromatography (HPLC) and radio-HPLC analyses were performed using a system that employs a Hewlett-Packard quaternary 1050 gradient pump, a Protein-Pak glass 300SW column (7.5 mm×300 mm, 10 μm; Waters Corp., Milford, MA), a variable wavelength UV detector and a Beckman 170 radiation detector. Gamma-ray spectroscopy was performed on a GEM18180-P HPGe detector coupled to a PC-based multichannel analyzer (AMETEK, Oak Ridge. TN). The spectra were analyzed using the Maestro-32 software (ORTEC, Oak Ridge, TN). Calibration setting numbers 72 and 92 on a Capintec CRC-55tR dose calibrator were used for freshly purified ²²⁷Th solutions and solutions 1 day after purification, respectively (Ferrier M G, Li Y. Chyan M K, et al. Thorium chelators for targeted alpha therapy: Rapid chelation of thorium-226. J Labelled Comp Radiopharm. 2020; 63:502-516. A Wallac Wizard 1470 gamma counter was also used to determine radiochemical purity of labeled products and count animal tissues.

The conjugation reaction followed a protocol similar to previously described (Price E W, Cawthray J F, Bailey G A. et al. H4octapa: an acyclic chelator for 111In radiopharmaceuticals. J Am Chem Soc. 2012; 134:8670-8683; Price E W, Zeglis B M, Cawthray J F, et al H(4)octapa-trastuzumab; versatile acyclic chelate system for 111In and 177Lu imaging and therapy. J Am Chem Soc. 2013; 135:12707-12721). Briefly, the conjugation reaction was conducted in HEPES buffer (50 mM HEPES, 150 mM NaCl, pH 8.5). The HEPES buffer was passed over a Chelex 100 (BioRad, USA) column to remove trace metals prior to use. Metal-free mAb (αGPC3 or αBHV1) was reacted with 5, 10 or 15 eq of octapa-NCS at room temperature (RT) with gentle mixing for about 16 h. Then the mixture was transferred to a Slide-A-Lyzer™ dialysis cassette and dialyzed against 4 L metal-free citrate buffer (50 mM citrate, 15 mM NaCl, pH 5.5) three times to quench the reaction. This was followed by dialysis against 3 L of metal-free saline changing twice per day for another 3 days. The octapa conjugates were then removed from the Slide-A-Lyzer™ dialysis cassette and stored under 4° C.

Isoelectric focusing (IEF) analyses were conducted on a XCell SureLock Mini-Cell Electrophoresis system using pH 3-10 IEF gels (Thermo Fisher Scientific, Waltham, MA) to determine how the isoelectric point (pI) of the antibodies changed because of the conjugation of octapa-NCS SERVA IEF standards used included: Cytochrome C; Pig heart origin; 10.7 pI. Ribonuclease A; Bovine pancreas origin; 9.5 pI, a Lectin; Lens culinaris origin; 8.3, 8.0, 7.8 pI, a Myoglobin; Horse muscle origin; 7.4, 6.9 pI, Carbonic anhydrase, Bovine erythrocytes origin; 6.0 pI, a B. Lactoglobulin; Bovine milk origin; 5.3, 5.2 pI. Trypsin inhibitor; Soybean origin; 4.5 pI. Glucose oxidase; Aspergillus niger origin; 4.2 pI, Amyloglucosidase; Aspergillus niger origin; 3.5 pI. Size exclusion (SE)-HPLC analyses were performed to assess the purity of the antibody conjugates. Mass spectral analyses of octapa-NCS conjugated mAbs were performed in duplicate on an Applied Biosciences SciEX 4800 MALDI TOF/TOF analyzer (Wellborn, TX) to estimate the average number of conjugates per αGPC3 or αBHV1 molecule.

### ²²⁷Th Radiolabeling of GPC3-Octapa

Before being used for radiolabeling reactions, ²²⁷Th was purified to remove its radioactive daughter, ²²³Ra. Briefly, ²²⁷Th was dissolved in 3M HNO₃ and H₂O₂, followed by heating to dryness. The residue was dissolved in 3M HNO₃ and the solution was loaded on a TEVA resin (Eichrom Technologies, Lisle, IL, USA). The column was rinsed with 3M, 1M and 0.5 M HNO₃, and the purified ²²⁷Th was eluted using 0.1 M HNO₃. Any purified ²²⁷Th that was not used within two days was purified again before use (Ferrier M G. Li Y, Chyan M K. et al. Thorium chelators for targeted alpha therapy: Rapid chelation of thorium-226. J Labelled Comp Radiopharm. 2020; 63:502-516). Antibody labeling conditions were optimized using small quantities of ²²⁷Th. In a typical reaction 200 μL of 0.05 M sodium citrate and 1 mM EDTA (pH 5.5) or 0.5 M ammonium acetate (NH₄OAc) (pH 5.5) was combined with 5-10 μL ²²⁷Th in 0.1 M HNO₃ (85.1-314.5 kBq. or 2.3-8.5 μCi). Metal-free sodium citrate buffer (1 M) was used to adjust the pH to 5-6.5, and subsequently 200-500 μg of αGPC3-octapa (4.0-5.7 mg/mL) was added and allowed to react for 30-120 min at RT or 37° C. The radiolabeling yield is also affected by the concentration of the ligand. The concentration dependence was not evaluated in this study but is of interest in future studies.

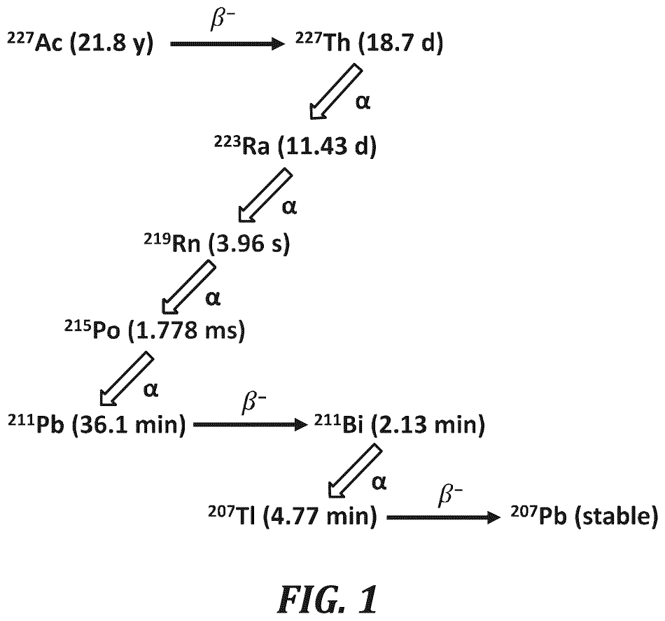

The GPC3-octapa conjugate that had the highest chelator-to-mAb ratio which maintained high antigen binding was used in the ²²⁷Th labeling reaction optimization. The decay scheme of ²²⁷Th is shown in FIG. 1. Optimization of the radiolabeling yield was accomplished by varying the reaction time, pH and temperature. Appropriate amounts of unlabeled mAb conjugates were added to the purified product to provide 0.5 μCi (18.5 kBq) and 70 μg antibody per dose. The remaining ²²⁷Th labeled GPC3-octapa was used for in vitro stability studies.

### ²²⁷Th Labeling of Antibodies for Animal Studies

To 200 μL of sodium citrate solution (0.05 M with 1 mM EDTA, pH 5.5), was added 60 μL of ²²⁷Th in 0.1 M HNO₃ (180-210 μCi, 6,660-7,770 kBq). The pH of the solution was adjusted to 5.5 by adding an appropriate amount of 1 M sodium citrate. It was allowed to react for 2 h at RI after 1 mg of αGPC-3-octapa (0.217 mL of 4.6 mg/mL) or αBHV1-octapa (0.21 mL of 4.75 mg/mL) was added to the reaction mixture. The ²²⁷Th labeled mAbs were purified using Econo-Pac® 10DG columns (BIO-RAD, Hercules, CA).

### In Vitro Stability of ²²⁷Th-Octapa-GPC3

Solutions of ²²⁷Th labeled mAbs were incubated at room temperature (RT) and pH 7.0 for 4 h, followed by refrigeration at 4° C., in the presence or absence of ascorbate acid. At 4 h, 24 h, 3 d, 7 d and 14 d time points, the percentage of protein-bound ²²⁷Th was determined by gamma-ray spectroscopy, monitoring the 236 keV (12.9%) gamma peak of ²²⁷Th. Free ²²⁷Th and antibody associated ²²⁷Th were separated by eluting 7-cm iTLC SG strips (Agilent, Santa Clara, CA) using 0.05 M sodium citrate with 1 mM EDTA (pH 5.5). The iTLC strips were cut into 3 sections. One 3-cm section that includes the origin to show activity bound to (denatured) protein, one 3-cm section that includes the solvent front to show free radioactivity, and a 1-cm section between the other two sections to determine if the radioactivity at the origin and at the solvent front were completely separated. The regions of the radio-iTLC strips that correspond to ²²⁷Th labeled αGPC3-octapa and free ²²⁷Th were measured on the HPGe detector using the same counting geometry. Although the percentage of protein-bound ²²⁷Th determined by gamma-ray spectroscopy was similar to that determined by automated gamma counter at 4 h. significant differences were observed for all later time points as the daughter ²²³Ra and other radioactive progenies could not be distinguished from ²²⁷Th by the automated gamma counter.

### Cell Lines and Tissue Culture

Cells were grown to 70-80% confluency, detached with 0.25% trypsin and passaged into new cell culture flasks per manufacturer instructions (PerkinElmer). Cells were passaged between 4-10 times between thawing and use in described experiments. Mycoplasma testing was not performed.

### Flow Cytometry

HepG2 cells were resuspended in cold phosphate-buffered saline (PBS) at a concentration of 1×10⁶ cells/mL. One microgram of unconjugated αGPC3, αGPC3-octapa or αBHV1-octapa was added to the cell suspension and incubated for 45 min at 4° C. After incubation, samples were washed in cold PBS, and incubated with 1 μg of FITC-labeled goat-α-mouse IgG₁ secondary antibody (Southern Biotech, cat. no. 1070-02) on ice for 30 min in the dark. Cells were then washed in cold PBS and were analyzed with a LSRII (BD Biosciences. San Jose, CA) using the FACS Diva software. A minimum of 10,000 cells were analyzed for each sample in triplicate. Data analysis was performed on the FlowJo software, version 8.8.6 (TreeStar, Ashland, OR; RRID:SCR_008520).

### Development of Orthotopic Xenograft Model

GPC3-positive HepG2-Red-FLuc (HepG2) cells expressing Luciferase were purchased from PerkinElmer (Bioware, cat. no. BW134280, RRID:CVCL_5198) and were maintained in a monolayer at 37° C. in Dulbecco's modified Eagle Medium (DMFM, Gibco) supplemented with 10% fetal bovine serum (FBS; Gibco) in a humidified chamber with 5% CO₂.

The orthotopic xenograft model was generated and described previously (Ludwig A D, et al. Yttrium-90-Labeled Anti-Glypican 3 Radioimmunotherapy Halts Tumor Growth in an Orthotopic Xenograft Model of Hepatocellular Carcinoma. J Oncol. 2019; 2019:4564707). Briefly, after a week of acclimatizing, 8-week-old female athymic Nu/J animals (20-25 g) (Jackson Laboratories) were anesthetized using 1.5% inhaled isoflurane and the left lobe of the liver was exposed through an upper midline laparotomy. Approximately 2×10⁶ Luciferase-expressing GPC3-positive HepG2 cells in 25 μL of Dulbecco's modified Eagle medium containing 50% Matrigel (BD Biosciences) were injected into the subcapsular space of the anterior left hepatic lobe. Four weeks after injection, a 75 mg/kg intraperitoneal injection of VivoGlo luciferin (Promega) was administered and bioluminescent imaging was performed using an IVIS Lumina II system (PerkinElmer) to verify tumor establishment.

To monitor orthotopic tumor growth, whole blood was obtained from animals by submandibular bleed and serum concentration of alpha-fetoprotein (AFP) was determined (Golde W T, et al. A rapid, simple, and humane method for submandibular bleeding of mice using a lancet. Lab Anim (NY). 2005; 34:39-43).

### Serum AFP Measurement

To monitor orthotopic tumor growth, whole blood was obtained from animals by submandibular bleed and collected in Eppendorf tubes (Golde W T, Gollobin P, Rodriguez L L. A rapid, simple, and humane method for submandibular bleeding of mice using a lancet. Lab Anim (NY). 2005; 34.39-43). Serum was extracted from the fresh whole blood and the serum concentration of alpha-fetoprotein (AFP) was determined on the UniCel Dxl 800 Access Immunoassay System (Beckman Coulter) using an Access AFP Alpha-fetoprotein pack (Quest Diagnostics; RRID:SCR_005210) and reported in nanograms/milliliter (ng/mL). Serum AFP has been extensively validated to correlate with tumor size in our model (Ludwig A D, Labadie K P, Seo Y D, et al. Yttrium-90-Labeled Anti-Glypican 3 Radioimmunotherapy Halts Tumor Growth in an Orthotopic Xenograft Model of Hepatocellular Carcinoma. J Oncol. 2019; 2019:4564707; Labadie K P, Ludwig A D, Lehnert A L, et al. Glypican-3 targeted delivery of (89)Zr and (90)Y as a theranostic radionuclide platform for hepatocellular carcinoma. Sci Rep. 2021; 11:3731).

### Biodistribution and Blood Clearance Studies

Tumor-bearing mice were injected with ²²⁷Th-octapa-αGPC3 or ²²⁷Th-octapa-αBHV1 (70 g, 500 kBq/kg). Tumors and normal organs were harvested 24 h, 7 d or 23 d after radioimmunoconjugate injection. Tissues were weighed and ²²⁷Th activity was measured by gamma counter, measured activity was a net sum of activity from all daughters. The percent injected dose of radioisotope per gram (% ID/g) of blood, tumor or organ was calculated after correcting for radioactive decay using an aliquot of the injectate, as were the tumor-to-normal organ ratios of absorbed radioactivity.

After tumor-bearing mice were injected with ^{cf}-octapa-αGPC3 or ²²⁷Th-octapa-αBHV1 (70 g, 500 kBq/kg), serial retro-orbital blood sampling was performed at 5, 15, 30, 60, 120, 240 min, and then collected on necropsy at 24 h, 7 d and 23 d after injection. Blood samples were measured by gamma counter and corrected for ²²⁷Th activity.

### ²²⁷Th Radioimmunotherapy

After animals were confirmed to have tumors by IVIS imaging, the animals were included in the study and serum AFP was measured. Tumor-bearing animals were assigned to one of four experimental groups based on serum AFP measurements to ensure comparable tumor burden among cohorts. In unblinded fashion, animals either received no treatment, αBHV1-octapa radiolabeled with 500 kBq/kg ²²⁷Th, αGPC3-octapa radiolabeled with 250 or 500 kBq/kg of ²²⁷Th via tail vein injection without anesthesia. Twenty-three days after injection, animals were euthanized to evaluate for early anti-tumor effect of ²²⁷Th-octapa-αGPC3 Serum was obtained for AFP measurement and randomly selected livers were harvested and placed in 10% (w/v) neutral-buffered formalin.

### Statistical Analysis

Statistical analysis was performed with GraphPad Prism (version 8.0.0, GraphPad Software. San Diego, California USA, RRID:SCR_002798). D'Agostino & Pearson normality test was performed to determine fit to a Gaussian distribution. Continuous variables were expressed as medians and means and compared by Student's t-test or Mann-Whitney test One way-ANOVA or Kruskal-Wallis with Dunn's multiple comparison test was performed. In all cases, a P value of 0.05 was considered statistically significant.

### Results

αGPC3 Conjugation with Octapa is Highly Efficient

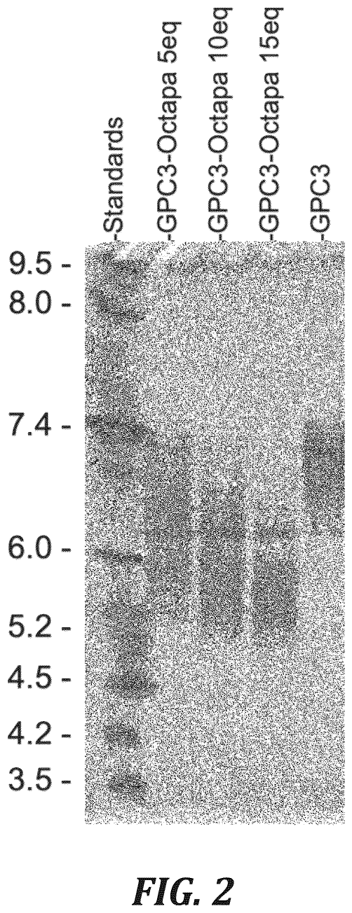

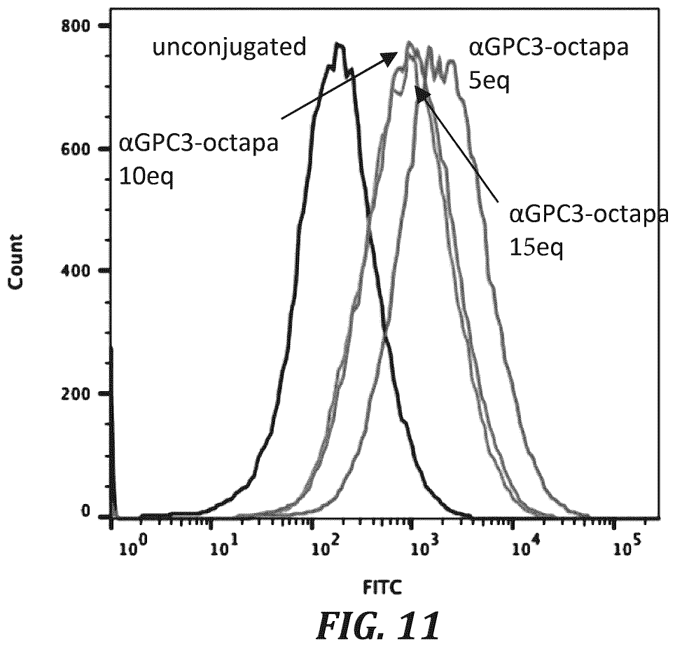

Octapa was conjugated to αGPC3 via reactions conducted using 5, 10 and 15 eq of octapa to determine the optimal conjugation ratio. The isoelectric points of the αGPC3-octapa shifted toward the acidic pI as the number of eq offered increased (FIG. 2). αGPC3-octapa 5 eq demonstrated minimum shift of binding to GPC3⁺ cells by flow cytometry (FIG. 11), however, it resulted in lower chelates per antibody compared to the 10 eq, therefore 10 eq conjugation ratio was used for subsequent experiments (FIG. 2). The protein recovery from the conjugation process was >85% and the final concentrations were 4.6 mg/mL and 4.75 mg/mL for αGPC3-octapa and αBHV1-octapa, respectively. Mass spectral analysis of αGPC3-octapa and αBHV1-octapa conjugates produced by reaction of 10 eq of octapa indicated that there was an average of 3.3 octapa moieties on αGPC3 and 5 octapa moieties on (BHV1.

### ²²⁷Th-Labeled αGPC3-Octapa is Stable In Vitro

Labeling conditions were optimized using αGPC3-octapa and small quantities of ²²⁷Th in 0.1 M HNO₃ (85.1-314.5 kBq. or 2.3-8.5 μCi). Labeling yield increased from 16%, 25% to 37% as the reaction time increased from 30 mm, 1 h to 2 h, respectively, but did not significantly increase after 2 h. Among the different reaction solutions and pH tested, the highest consistent yields were obtained when 200 μL of 0.05 M sodium citrate with 1 mM EDTA (pH 5.5) was combined with 5-10 μL ²²⁷Th in 0.1 M HNO₃ with the pH adjusted to 5-5.5 using 1 M sodium citrate. To this, 200 μg of αGPC3-octapa (4.0 mg/mL) was added and allowed to react for 2 h at 37° C. (43-70% radiochemical yield). Reactions conducted at the elevated temperature only accounted for about 4% higher yields, therefore subsequent reactions were conducted at RT.

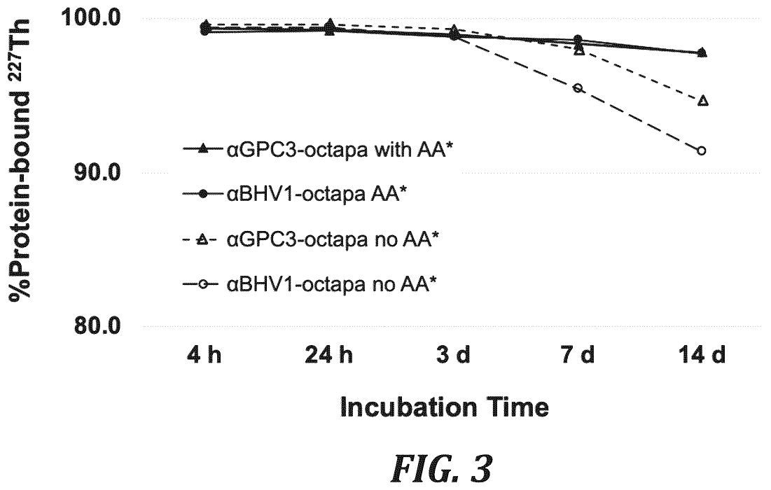

In vitro stability of the ²²⁷Th-labeled mAb-octapa conjugates was evaluated with and without ascorbic acid. Because the gamma counter cannot distinguish ²²⁷Th activity from its radioactive progeny, gamma spectroscopy was used to analyze radio-iTLC strips and determine the percentage of antibody-bound ²²⁷Th. After 14 d, ≥97.8% of 227Th was observed to be bound to GPC3-octapa in the presence of ascorbic acid (FIG. 3).

### GPC3-Octapa Maintains Ligand Binding In Vitro and In Vivo

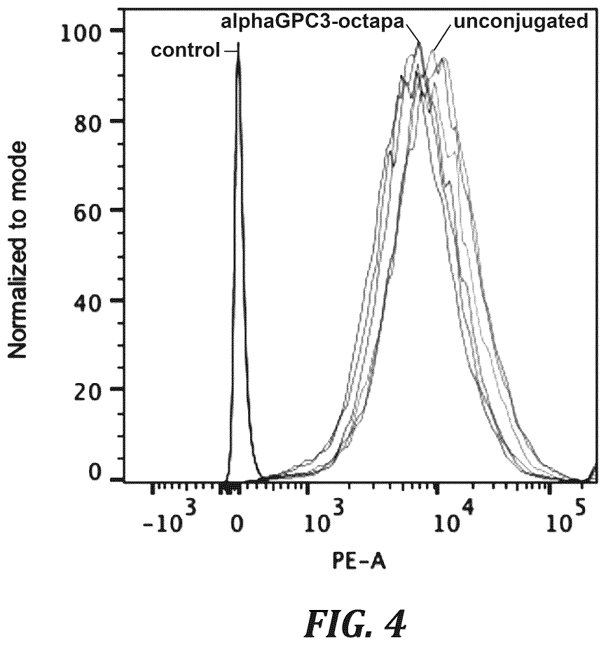

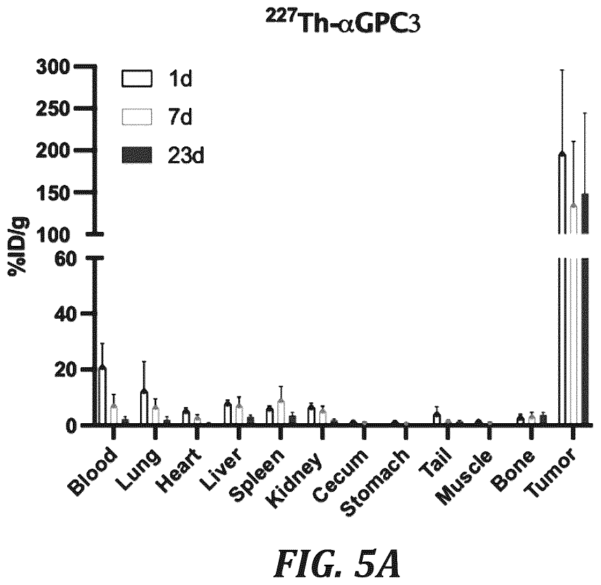

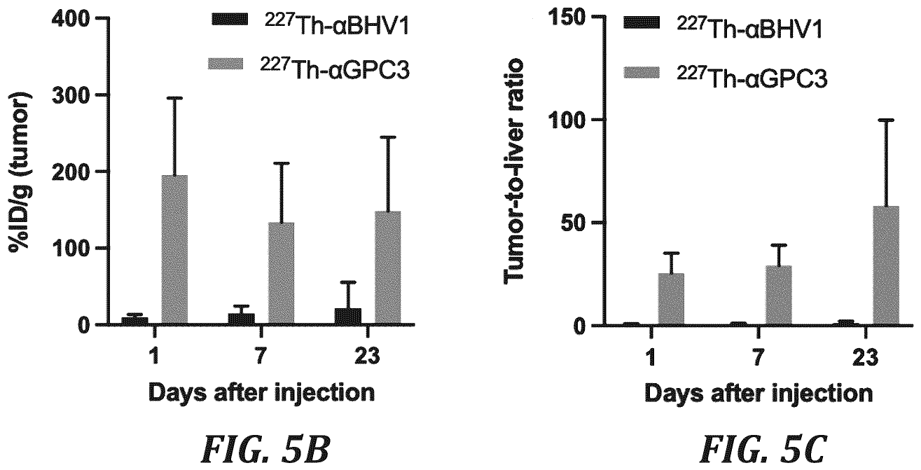

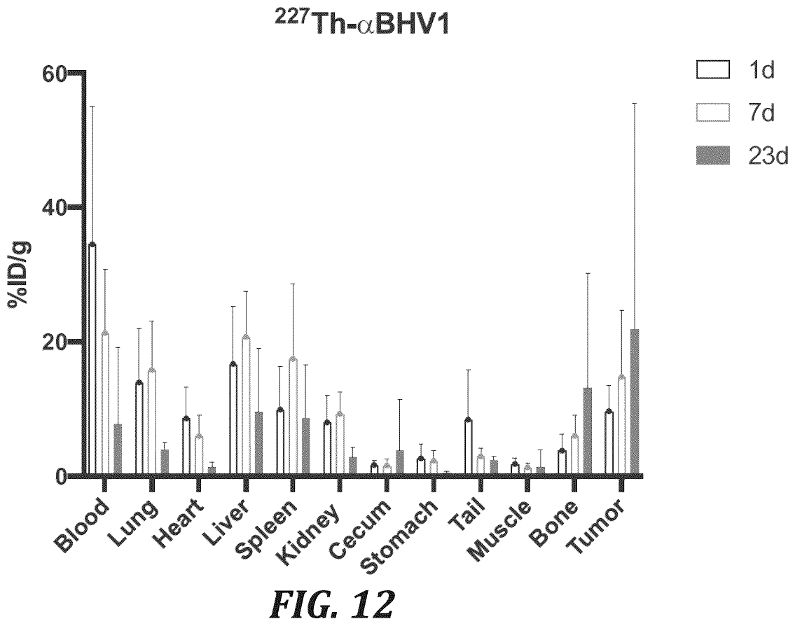

The affinity for GPC3 by αGPC3-octapa was assessed with flow cytometry using HepG2 cells. GPC3-octapa maintained high affinity for GPC3 with only modest reduction in binding affinity compared to unconjugated αGPC3 (FIG. 4). In tumor-bearing mice 24 h, 7 d and 23 d after tail vein injection of ²²⁷Th-octapa-αGPC3, high levels of radioactivity were detected in the tumor tissue compared to surrounding liver and other organs (FIG. 5A, Table 1) % ID/g of ²²⁷Th-octapa-αGPC3 remained high in tumor tissue over 23 d (FIG. 5B). The ratio of % ID/g of ²²⁷Th-octapa-αGPC3 in tumor tissue compared to adjacent normal liver parenchyma steadily increased over time (FIG. 5C). ²²⁷Th-octapa-αBHV1, an irrelevant isotype antibody conjugate, did not significantly bind to the tumor tissue as expected (FIG. 12).

TABLE 1
Tumor to organ ratios after injection of 500 kBq/kg
²²⁷Th-αGPC3 at 1, 7 and 23 days after injection.

| Tumor | Day 1 | | Day 7 | | Day 23 | |
|---|---|---|---|---|---|---|
| Organ ratio | Avg | Stdev | Avg | Stdev | Avg | Stdev |
| Blood | 12.0 | 6.8 | 60.9 | 58.9 | 90.8 | 70.0 |
| Lung | 23.4 | 18.6 | 26.9 | 5.6 | 118.4 | 80.7 |
| Heart | 46.4 | 27.4 | 80.2 | 34.3 | 301.1 | 191.5 |
| Liver | 25.7 | 9.5 | 29.3 | 9.7 | 58.3 | 41.5 |
| Spleen | 35.7 | 19.2 | 18.7 | 10.5 | 49.1 | 31.6 |
| Kidney | 29.1 | 7.2 | 31.1 | 8.5 | 101.5 | 70.5 |
| Cecum | 163.5 | 62.8 | 356.9 | 312.7 | 480.0 | 306.3 |
| Stomach | 166.6 | 77.6 | 360.9 | 216.8 | 794.2 | 590.6 |
| Tail | 52.3 | 33.3 | 115.8 | 47.2 | 115.8 | 77.3 |
| Muscle | 133.7 | 61.8 | 776.9 | 872.1 | 829.5 | 734.2 |
| Bone | 96.2 | 50.5 | 76.6 | 40.2 | 46.9 | 40.8 |

²²⁷Th-octapa-αGPC3 did not significantly accumulate in normal tissues at 1, 7 and 23 d after injection (FIG. 5A). % ID/g of ²²⁷*h-octapa-αGPC3 was <5% in all tested normal tissues by day 23 after injection. The primary mode of decay for ²²⁷Th is alpha decay, resulting in daughter radium-223 particles, a radionuclide which preferentially accumulates in bone. Measurement of radioactivity of the femur demonstrated low gamma counts after injection with ²²⁷Th-octapa-αGPC3 compared to ²²⁷Th-octapa-αBHV1, where radioactivity was observed to accumulate in bone over time. This is presumably due to the highly specific targeting and preferential accumulation of ²²⁷Th-octapa-αGPC3 in the tumor, resulting in lower circulating radioactivity for bone deposition. High-resolution gamma-ray spectroscopy was performed on select tissues demonstrating radium accumulation in bone (Table 2), but further evaluation will be part of future study.

TABLE 2
Distribution of 223Ra and 227Th in tissues. 223Ra/227Th
ratios and normalized 223Ra/227Th ratios in selected
tissues 21 days after injection of of 500 kBq/kg 227Th-
αGPC3 determined by high-resolution gamma-ray spectroscopy.
The standard is an aliquot of the injectate.

| | ²²³Ra/²²⁷Th ratio | Normalized ²²³Ra/²²⁷Th |
|---|---|---|
| Standard | 0.78 | 1.00 |
| Bone | 2.86 | 3.67 |
| Kidneys | 0.29 | 0.37 |
| Liver | 0.37 | 0.48 |
| Spleen | 0.56 | 0.71 |
| Tumor | 0.30 | 0.38 |

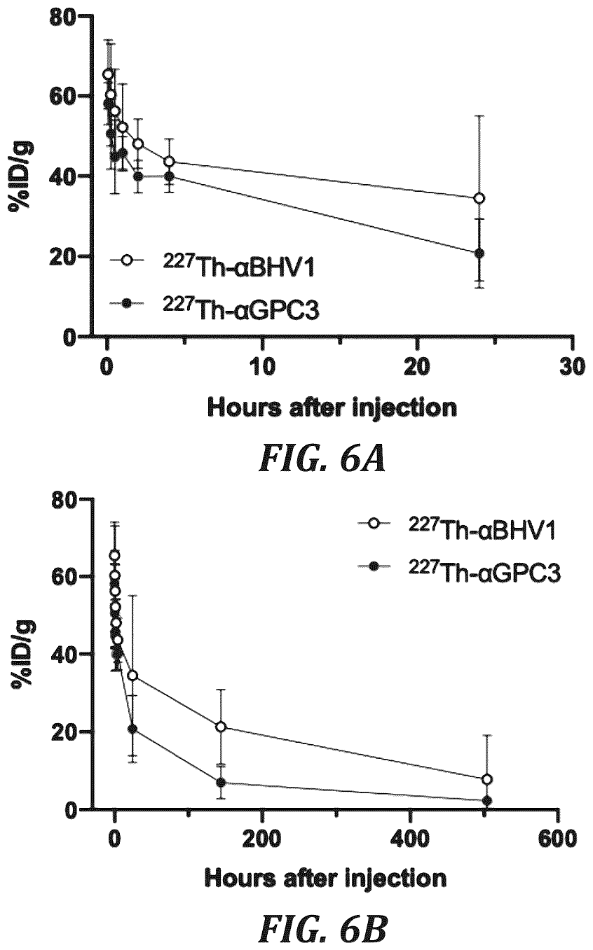

Blood radioactivity was more rapidly cleared after ²²⁷Th-octapa-αGPC3 injection compared to ²²⁷Th-octapa-αBHV1 with serum half-life of 14 and 17 hours, respectively. This more rapid clearance may be secondary to increased accumulation of the radioimmunoconjugate in the tumor over time (FIG. 6).

### ²²⁷Th-Octapa-αGPC3 Reduces Tumor Burden in Murine Model

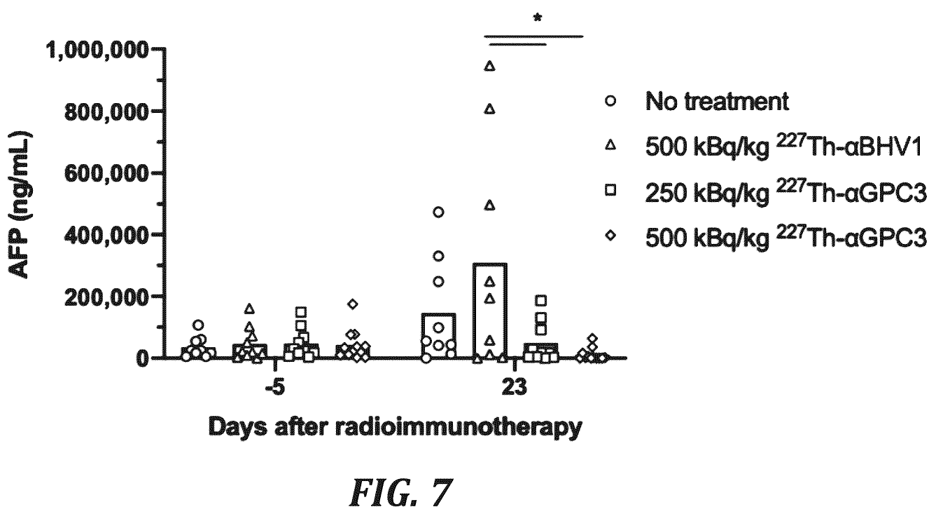

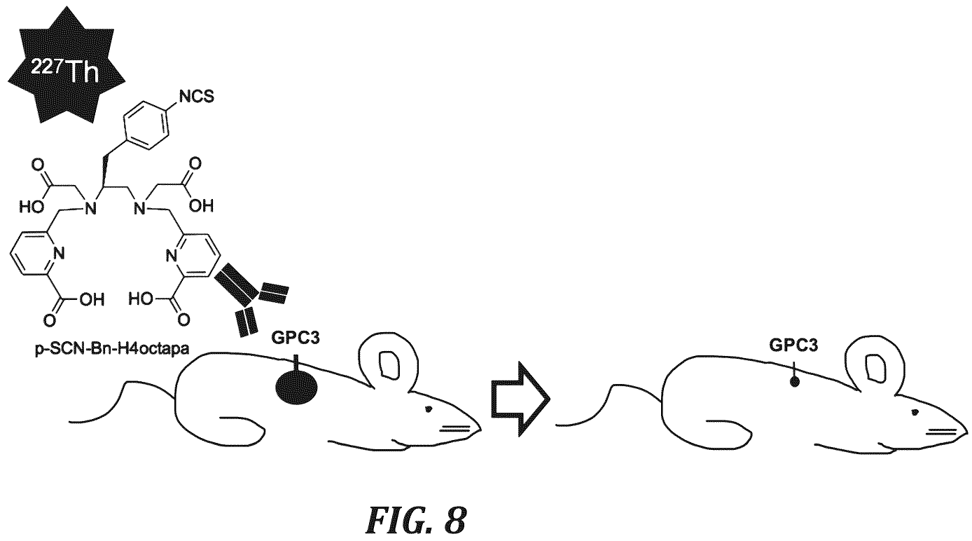

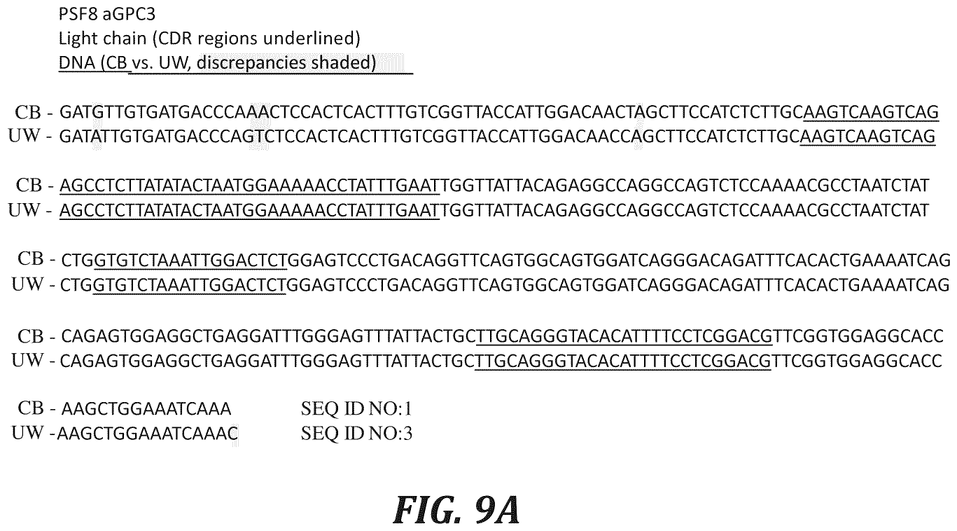

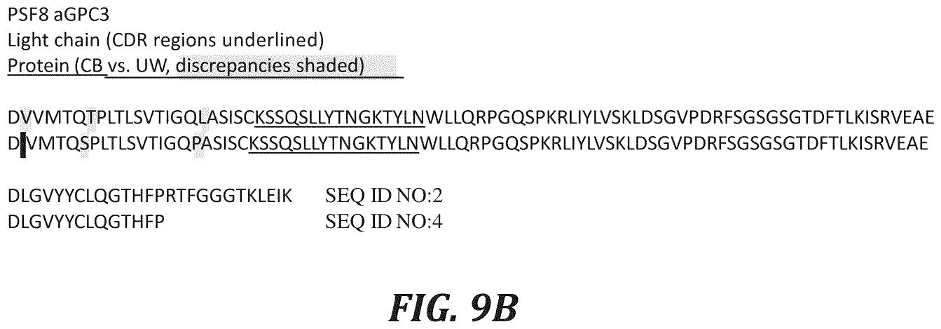

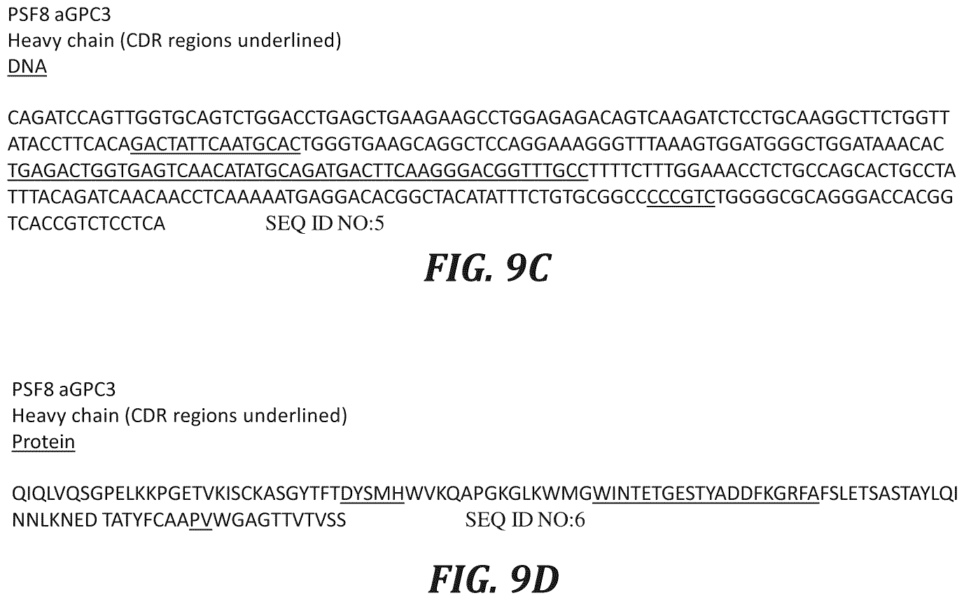

To assess efficacy of ²²⁷Th-octapa-αGPC3 TAT, tumor-bearing mice received either no treatment, 500 kBq/kg ²²⁷Th-octapa-αBHV1, 250 or 500 kBq/kg ²²⁷Th-octapa-αGPC3 by tail vein injection. Serum AFP was significantly lower in mice treated with ²²⁷Th-octapa-αGPC3 compared to control groups 23 d after therapy administration (FIG. 7). The treatment effect was most pronounced after therapy with 500 kBq/kg ²²⁷Th-octapa-αGPC3, although a modest effect was observed after therapy with 250 kBq/kg. AFP increased significantly after therapy with 500 kBq/kg ²²⁷Th-octapa-αBHV1, indicating that GPC3 targeted thorium delivery induced tumor cell killing rather than the presence of systemically circulating antibody-bound ²²⁷Th.

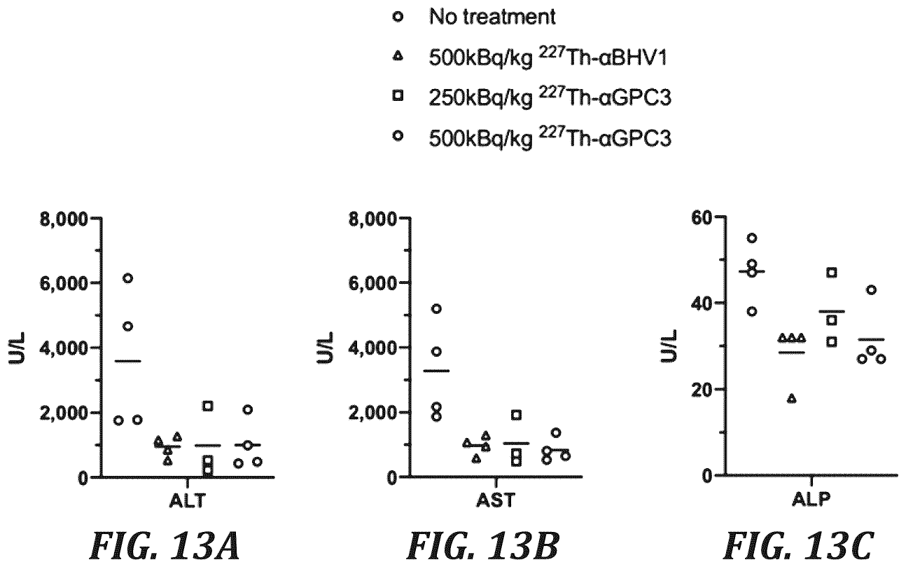

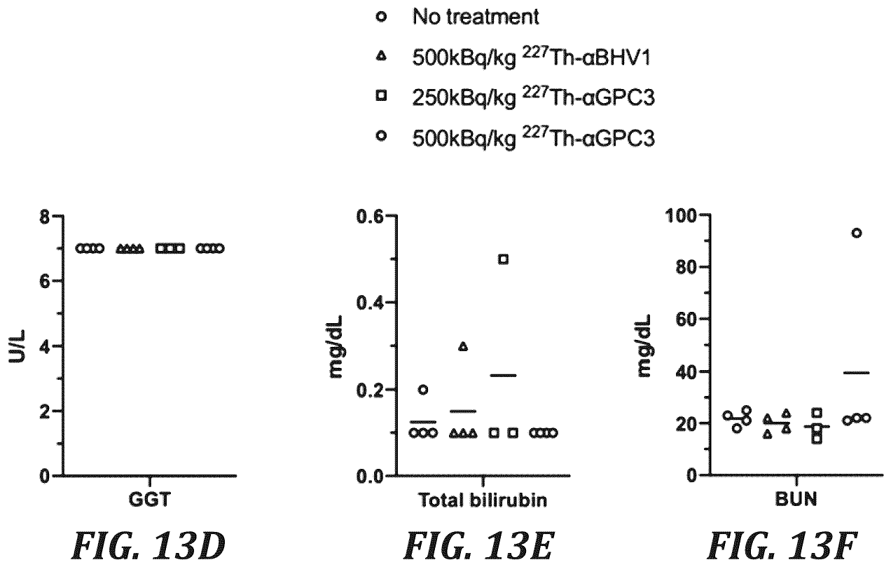

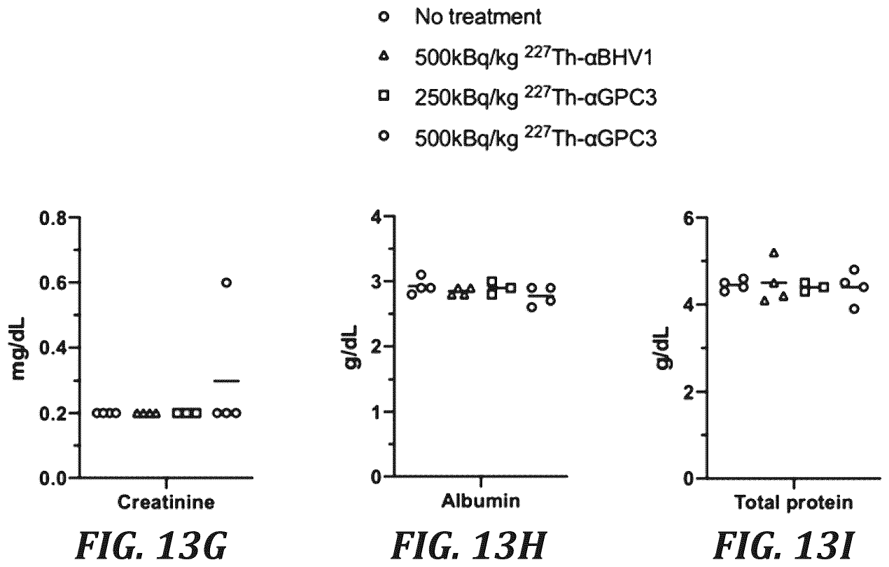

To assess for organ-specific toxicity after administration of the ²²⁷Th radioimmunoconjugates, serum markers of end organ dysfunction were collected 23 d after injection, and no significant aberrations were identified in comparison to control (FIG. 13). No animals died prior to termination of study.

### Discussion

In this Example, the development of a thorium-227 radioimmunoconjugate targeting GPC3 and demonstration of its in vivo efficacy in the treatment of HCC was described in an orthotopic murine xenograft model. The ²²⁷Th-octapa-αGPC3 radioimmunoconjugate was observed to be stable in vitro, maintain its specificity for GPC3 with a favorable biodistribution, and result in tumor reduction without undesired significant acute toxicity. These findings add to previous studies establishing the basis for a GPC3 targeted theranostic platform whereby different radioimmunoconjugates can be used for diagnostic/surveillance imaging and treatment. Such a platform can improve current treatments of HCC by enabling earlier identification of disease or recurrence, increase the accuracy of staging, and allow for more targeted treatment with less systemic toxicities.

To radiolabel the antibody described here, a picolinic acid (pa)-containing chelate, octapa, an octadentate acyclic ligand was utilized that enables ²²⁷Th radiolabeling of antibodies at RT, helping to maintain the three-dimensional conformation and immunoreactivity of the conjugated targeting antibody. Optimization of the reaction conditions resulted in efficient ²²⁷Th-labeling (up to 70% radiolabeling yield) and product with high radiochemical purity. The high in vivo stability of the ²²⁷Th-octapa complex is demonstrated by the low bone uptake throughout the 23-day study period. In its initial description, H4octapa was used to label trastuzumab with indium-111 and lutetium-177 for imaging and therapy, respectively, of mice bearing ovarian cancer xenografts. Previously ²²⁷Th has been radiolabeled to antibodies using an octadentate hydroxypyridino (HOPO) for the treatment of CD33 myeloid leukemia, CD70 renal cell carcinoma and mesothelin-positive malignancies. This Example demonstrates that pa ligands can be used as a new class of ligand for ²²⁷Th radiolabeling of antibody conjugates.

The inventors have previously described conjugating GPC3 with DOTA chelate for yttrium-90 (⁹⁰Y-GPC3) radioimmunotherapy/Yttrium-90 produces beta ionizing radiation, with lower energy and longer path length compared to ₂₂₇Th, which produces alpha ionizing radiation. These differences in properties are important as higher energy transfer results in a lower LD₅₀, and a shorter path length decreases the radius of tissues affected by the radiation. Alpha therapies can be desirable over beta therapies if highly specific targeting is possible. While research into TAT for hematologic malignancies has focused on ₂₁₁At (t_{1/2} 7.21 h), and a recent study for HCC described using actinium-225 (₂₂₅Ac, t_{1/2} 9.92 d), the inventors elected to use ²²⁷Th (t_{1/2} 18.7 d) for its longer half-life which can be advantageous in the treatment of solid tumors.

Administration of ²²⁷Th-octapa-αGPC3 led to highly specific tumor uptake, rapid blood clearance and robust antitumor activity without significant acute toxicity. Within one day of injection of ²²⁷Th-octapa-αGPC3, significant intra-tumoral accumulation was observed compared to control. Tumor to liver ratio of ²²⁷Th-octapa-αGPC3 steadily increased over time and minimal off-target uptake was observed, indicating highly specific targeting and clearance. Modest bone uptake was observed over time in the irrelevant isotype antibody control group, which is expected given ²²⁷Th daughter molecule ²²³Ra delivers radiation to sites of increased osteoblastic metabolism.

The observed therapeutic effect of ²²⁷Th-octapa-αGPC3 was dependent on antibody targeted delivery of radiation, as evidenced by the lack of therapeutic effect observed in the nontargeting ²²⁷Th-octapa-αBHV1 control group. Using an established marker of tumor burden in the disclosed model, serum AFP, the therapeutic effect after treatment with 500 kBg/kg ²²⁷Th-octapa-αGPC3 to be consistent, with a reduction in serum AFP in all animals except one. Interestingly, a marked increase in tumor burden in the irrelevant antibody control group was observed. The mechanism of this finding is not understood but could be related to bone marrow toxicity and suppression of alloreactive immune cells, which are present in athymic mice.

No significant acute off-target toxicity was observed in our study with all animals surviving until study completion. There was moderate amount of radioactivity identified in the bone, particularly in our control group. One of the challenges of using alpha particle emitters for therapy is the presence of multiple radioactive daughter products that may dislocate from target site. Although this may lead to cytopenias and marrow toxicity, data from human trials with similar radioisotopes are reassuring. In the phase 3 ALSYMPACA trial, difference in cytopenia rates were seen in patients with prior docetaxel dosing, suggesting differences in cumulative marrow damage being more implicated over direct radioactive effects. A recently published manuscript describing GPC3 TAT using ²²⁵Ac conjugated to the humanized monoclonal antibody GC33 in a heterotopic murine xenograft model demonstrated modest anti-tumor activity while observing significant bone marrow suppression and toxicity. We postulate that the difference in toxicity between our studies is due to improved specificity of our antibody highlighting the importance of effective targeting for delivery of the alpha therapy, and the advantage of an orthotopic liver xenograft model compared to a subcutaneous xenograft model.

GPC3 expression in HCC is variable and differs based on the degree of differentiation. Human HepG2 cells demonstrate high expression of GPC3, and may not recapitulate lower to intermediate grade HCC. The studies were performed in athymic mice that lack mature T-cells. Although competent leukocytes are present in this model, it likely does not represent the complex tumor microenvironment of human HCC. We elected to omit a non-radiolabeled antibody conjugate control group due to extensive prior work by our group demonstrating that GPC3 antibody alone does not lead to robust antitumor response. Tumor size was measured indirectly in our model by serum AFP. Direct tumor size measurements via ultrasonography or bioluminescent imaging was performed due to environmental health and safety constraints at our core facilities with radioactive animals. Some untreated animals observed spontaneous reduction in AFP and presumably tumor size without treatment, possibly secondary to an alloreactive response from native immune cells. While these animals existed in all groups, this requires further investigation. More studies are warranted to evaluate the potential toxicity of ²²⁷Th-octapa-αGPC3. One of the studies would be to perform dosimetry analysis to understand the radiation dose from ²²⁷Th and its alpha-emitting decay progenies, especially ²²³Ra. Additionally, it is worth noting that radiation nephropathy and other toxicities were not able to be fully assessed with only 23 days of monitoring. A longer period of observation in addition to hematologic analysis, which was not performed due lack of appropriate experimental equipment, is planned for future investigations.

### CONCLUSION

In conclusion, we report the development of a GPC3-targeted ²²⁷Th conjugate using octapa and demonstrate it to be stable. In vitro, maintain high specificity for GPC3 with favorable biodistribution in vivo, and result in significant antitumor activity without undesirable acute toxicity. To our knowledge, this is the first description of a thorium-227 radiopharmaceutical targeting GPC3 and is a promising addition to the theranostic approach to treating HCC. Thorium was reliably and efficiently labeled to a glypican-3 targeting antibody via octapa chelator. This radioimmunoconjugate maintained affinity for the target antigen in vitro and in vivo. Significant levels of thorium accumulated intratumorally. Orthotopic mice treated with glypican-3 directed thorium therapy had significant reductions in their tumor burden compared to control animals. This study identifies a new approach to treating HCC using a personalized and targeted approach against a highly expressed antigen in HCC.

TABLE 3
Amino acid and nucleotide sequence

| Construct/sequence | SEQ ID NO: |
|---|---|
| P5F8CB αGPC3 Variable Light Chain (V_{L}) region | 1 |
| GATGTTGTGATGACCCAAACTCCACTCACTTTGTCGGTTACCATTGGACAAC | |
| TAGCTTCCATCTCTTGCAAGTCAAGTCAGAGCCTCTTATATACTAATGGAAA | |
| AACCTATTTGAATTGGTTATTACAGAGGCCAGGCCAGTCTCCAAAACGCCT | |
| AATCTATCTGGTGTCTAAATTGGACTCTGGAGTCCCTGACAGGTTCAGTGG | |
| CAGTGGATCAGGGACAGATTTCACACTGAAAATCAGCAGAGTGGAGGCTG | |
| AGGATTTGGGAGTTTATTACTGCTTGCAGGGTACACATTTTCCTCGGACGTT | |
| CGGTGGAGGCACCAAGCTGGAAATCAAA | |
| P5F8CB αGPC3 Variable Light Chain (V_{L}) region | 2 |
| DVVMTQTPLTLSVTIGQLASISCKSSQSLLYTNGKTYLNWLLQRPGQSPKRLIY | |
| LVSKLDSGVPDRFSGSGSGTDFTLKISRVEAEDLGVYYCLQGTHFPRTFGGGT | |
| KLEIK | |
| P5F8UW αGPC3 Variable Light Chain (V_{L}) region | 3 |
| GATATTGTGATGACCCAGTCTCCACTCACTTTGTCGGTTACCATTGGACAA | |
| CCAGCTTCCATCTCTTGCAAGTCAAGTCAGAGCCTCTTATATACTAATGGA | |
| AAAACCTATTTGAATTGGTTATTACAGAGGCCAGGCCAGTCTCCAAAACG | |
| CCTAATCTATCTGGTGTCTAAATTGGACTCTGGAGTCCCTGACAGGTTCAG | |
| TGGCAGTGGATCAGGGACAGATTTCACACTGAAAATCAGCAGAGTGGAGG | |
| CTGAGGATTTGGGAGTTTATTACTGCTTGCAGGGTACACATTTTCCTCGGA | |
| CGTTCGGTGGAGGCACCAAGCTGGAAATCAAAC | |
| P5F8UW αGPC3 Variable Light Chain (V_{L}) region | 4 |
| DIVMTQSPLTLSVTIGQPASISCKSSQSLLYTNGKTYLNWLLQRPGQSPKRLIY | |
| LVSKLDSGVPDRFSGSGSGTDFTLKISRVEAEDLGVYYCLQGTHFP | |
| P5F8 αGPC3 Variable Heavy Chain (V_{H}) region | 5 |
| CAGATCCAGTTGGTGCAGTCTGGACCTGAGCTGAAGAAGCCTGGAGAGAC | |
| AGTCAAGATCTCCTGCAAGGCTTCTGGTTATACCTTCACAGACTATTCAAT | |
| GCACTGGGTGAAGCAGGCTCCAGGAAAGGGTTTAAAGTGGATGGGCTGGA | |
| TAAACACTGAGACTGGTGAGTCAACATATGCAGATGACTTCAAGGGACGG | |
| TTTGCCTTTTCTTTGGAAACCTCTGCCAGCACTGCCTATTTACAGATCAACA | |
| ACCTCAAAAATGAGGACACGGCTACATATTTCTGTGCGGCCCCCGTCTGGG | |
| GCGCAGGGACCACGGTCACCGTCTCCTCA | |
| P5F8 αGPC3 Variable Heavy Chain (V_{H}) region | 6 |
| QIQLVQSGPELKKPGETVKISCKASGYTFTDYSMHWVKQAPGKGLKWMGWI | |
| NTETGESTYADDFKGRFAFSLETSASTAYLQINNLKNEDTATYFCAAPVWGA | |
| GTTVTVSS | |
| P5F8 αGPC3 V_{L} CDR1 | 7 |
| AAGTCAAGTCAGAGCCTCTTATATACTAATGGAAAAACCTATTTGAAT | |
| P5F8 αGPC3 V_{L} CDR1 | 8 |
| KSSQSLLYINGKTYLN | |
| P5F8 αGPC3 V_{L} CDR2 | 9 |
| GTGTCTAAATTGGACTCT | |
| P5F8 αGPC3 V_{L} CDR2 | 10 |
| VSKLDS | |
| P5F8 αGPC3 V_{L} CDR3 | 11 |
| TTGCAGGGTACACATTTTCCTCGGACG | |
| P5F8 αGPC3 V_{L} CDR3 | 12 |
| LQGTHFPRT | |
| P5F8 αGPC3 V_{H} CDR1 | 13 |
| GACTATTCAATGCAC | |
| P5F8 αGPC3 V_{H} CDR1 | 14 |
| DYSMH | |
| P5F8 αGPC3 V_{H} CDR2 | 15 |
| TGGATAAACACTGAGACTGGTGAGTCAACATATGCAGATGACTTCAAGGG | |
| ACGGTTTGCC | |
| P5F8 αGPC3 V_{H} CDR2 | 16 |
| WINTETGESTYADDFKGRFA | |
| P5F8 αGPC3 V_{H} CDR3 | |
| CCCGTC | |
| P5F8 αGPC3 V_{H} CDR3 | |
| PV | |

While illustrative embodiments have been illustrated and described, it will be appreciated that various changes can be made therein without departing from the spirit and scope of the invention.

1. An antibody, comprising:
a light chain variable domain comprising:
a CDRL1 with an amino acid sequence as set forth in SEQ ID NO:8;
a CDRL2 with an amino acid sequence as set forth in SEQ ID NO:10; and
a CDRL3 with an amino acid sequence as set forth in SEQ ID NO:12; and
a heavy chain variable domain comprising:
a CDRH1 with an amino acid sequence as set forth in SEQ ID NO:4;
a CDRH2 with an amino acid sequence as set forth in SEQ ID NO:16; and
a CDRH3 with an amino acid sequence as set forth in SEQ ID NO:18;
wherein the antibody specifically binds glypican-3 (GPC3).

2. The antibody of claim 1, wherein the antibody comprises a variable light chain region comprising an amino acid sequence with at least 90% identity to the sequence set forth in SEQ ID NO:2 or SEQ ID NO:4.

3. The antibody of claim 1, wherein the antibody comprises a variable heavy chain region comprising an amino acid sequence with at least 90% identity to the sequence set forth in SEQ ID NO:6.

4. The antibody of claim 1, wherein the antibody is a monoclonal antibody.

5. The antibody of claim 1, wherein the antibody is a humanized antibody.

6. The antibody of claim 1, wherein the antibody is conjugated to a radioisotope.

7. The antibody of claim 6, wherein the radioisotope is thorium-227.

8. The antibody of claim 7, wherein the thorium-227 is conjugated to the antibody via p-SCN-BN-H₄octapa-NCS.

9. The antibody of claim 1, wherein the antibody is conjugated to p-SCN-BN-H₄octapa-NCS.

10. A pharmaceutical composition comprising the antibody according to claim 1 and a pharmaceutically acceptable carrier.

11. A nucleic acid molecule comprising a nucleotide sequence encoding the amino acid sequence of the antibody according to claim 1.

12. The nucleic acid molecule of claim 11, characterized by one or more of the following:
wherein the CDRL1 is encoded by a nucleic acid sequence as set forth in SEQ ID NO:7;
wherein the CDRL2 is encoded by a nucleic acid sequence as set forth in SEQ ID NO:9;
wherein the CDRL3 is encoded by a nucleic acid sequence as set forth in SEQ ID NO:11;
wherein the CDRH1 is encoded by a nucleic acid sequence as set forth in SEQ ID NO:13;
wherein the CDRH2 is encoded by a nucleic acid sequence as set forth in SEQ ID NO:15; or
wherein the CDRH3 is encoded by a nucleic acid sequence as set forth in SEQ ID NO:17.

13. A vector comprising the nucleic acid of claim 11 and a promoter operatively linked to the nucleic acid.

14. A method of detecting glypican-3 (GPC3) on a cell or tissue, the method comprising contacting the cell or tissue with the antibody of claim 1 and detecting binding of the antibody to the cell or tissue.

15. The method of claim 14, wherein the cell is a liver cell or the tissue is a liver tissue.

16. The method of claim 14, wherein the cell or tissue is in vitro in a biological sample obtained from a subject.

17. The method of claim 14, wherein the cell or tissue is in vivo in a subject.

18. The method of claim 16, wherein detection of binding of the antibody to the cell or tissue indicates that the subject has hepatocellular carcinoma (HCC).

19. A method of imaging the presence or distribution of glypican-3 (GPC3) in a subject, the method comprising administering the pharmaceutical composition of claim 10 to the subject.

20. A method of treating a hepatocellular carcinoma (HCC) in a subject in need thereof, the method comprising administering an effective amount of the pharmaceutical composition of claim 10 to the subject.

21. (canceled)


SEQUENCE LISTING (16 sequences)

SEQ ID NO: 1 - DNA, 336 bp, unidentified
  source 1..336: mol_type = other DNA
gatgttgtgatgacccaaactccactcactttgtcggttaccattggacaactagcttccatctcttgcaagtcaagtcagagcctcttatatactaatggaaaaacctatttgaattggttattacagaggccaggccagtctccaaaacgcctaatctatctggtgtctaaattggactctggagtccctgacaggttcagtggcagtggatcagggacagatttcacactgaaaatcagcagagtggaggctgaggatttgggagtttattactgcttgcagggtacacattttcctcggacgttcggtggaggcaccaagctggaaatcaaa

SEQ ID NO: 2 - protein, 112 aa, synthetic construct
DVVMTQTPLTLSVTIGQLASISCKSSQSLLYTNGKTYLNWLLQRPGQSPKRLIYLVSKLDSGVPDRFSGSGSGTDFTLKISRVEAEDLGVYYCLQGTHFPRTFGGGTKLEIK

SEQ ID NO: 3 - DNA, 337 bp, unidentified
  source 1..337: mol_type = other DNA
gatattgtgatgacccagtctccactcactttgtcggttaccattggacaaccagcttccatctcttgcaagtcaagtcagagcctcttatatactaatggaaaaacctatttgaattggttattacagaggccaggccagtctccaaaacgcctaatctatctggtgtctaaattggactctggagtccctgacaggttcagtggcagtggatcagggacagatttcacactgaaaatcagcagagtggaggctgaggatttgggagtttattactgcttgcagggtacacattttcctcggacgttcggtggaggcaccaagctggaaatcaaac

SEQ ID NO: 4 - protein, 100 aa, synthetic construct
DIVMTQSPLTLSVTIGQPASISCKSSQSLLYTNGKTYLNWLLQRPGQSPKRLIYLVSKLDSGVPDRFSGSGSGTDFTLKISRVEAEDLGVYYCLQGTHFP

SEQ ID NO: 5 - DNA, 333 bp, unidentified
  source 1..333: mol_type = other DNA
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgagtcaacatatgcagatgacttcaagggacggtttgccttttctttggaaacctctgccagcactgcctatttacagatcaacaacctcaaaaatgaggacacggctacatatttctgtgcggcccccgtctggggcgcagggaccacggtcaccgtctcctca

SEQ ID NO: 6 - protein, 111 aa, synthetic construct
QIQLVQSGPELKKPGETVKISCKASGYTFTDYSMHWVKQAPGKGLKWMGWINTETGESTYADDFKGRFAFSLETSASTAYLQINNLKNEDTATYFCAAPVWGAGTTVTVSS

SEQ ID NO: 7 - DNA, 48 bp, unidentified
  source 1..48: mol_type = other DNA
aagtcaagtcagagcctcttatatactaatggaaaaacctatttgaat

SEQ ID NO: 8 - protein, 16 aa, synthetic construct
KSSQSLLYTNGKTYLN

SEQ ID NO: 9 - DNA, 18 bp, unidentified
  source 1..18: mol_type = other DNA
gtgtctaaattggactct

SEQ ID NO: 10 - protein, 6 aa, synthetic construct
VSKLDS

SEQ ID NO: 11 - DNA, 27 bp, unidentified
  source 1..27: mol_type = other DNA
ttgcagggtacacattttcctcggacg

SEQ ID NO: 12 - protein, 9 aa, synthetic construct
LQGTHFPRT

SEQ ID NO: 13 - DNA, 15 bp, unidentified
  source 1..15: mol_type = other DNA
gactattcaatgcac

SEQ ID NO: 14 - protein, 5 aa, synthetic construct
DYSMH

SEQ ID NO: 15 - DNA, 60 bp, unidentified
  source 1..60: mol_type = other DNA
tggataaacactgagactggtgagtcaacatatgcagatgacttcaagggacggtttgcc

SEQ ID NO: 16 - protein, 20 aa, synthetic construct
WINTETGESTYADDFKGRFA